(19) United States
(12) Patent Application Publication
(10) Pub. No.: US 2015/0066437 A1
(43) Pub. Date: Mar. 5, 2015

(54) GRAPHIC PRODUCT GENERATOR

(71) Applicant: Siemens Aktiengesellschaft, Munich (DE)

(72) Inventors: Moritz ONKEN, Nuernberg (DE); Jochen STICH, Nuernberg (DE)

(21) Appl. No.: 14/471,029

(22) Filed: Aug. 28, 2014

(30) Foreign Application Priority Data
Aug. 30, 2013 (DE) 102013217350.4

(51) Int. Cl. G01D 21/00; A61B 6/00; G01R 35/00

(52) U.S. Cl. CPC G01D 21/00; G01R 35/00; A61B 6/586
USPC 702/189

(57) ABSTRACT

A method, a checking system, a computer program product and a control computer are disclosed for checking a configuration for a multi-component computed tomography device. After a change of a configuration setting, a targeted check of device components can be carried out through created rules and index data.

### PRIORITY STATEMENT

The present application hereby claims priority under 35 U.S.C. §119 to German patent application number DE 102013 217350.4 filed Aug. 30, 2013, the entire contents of which are hereby incorporated herein by reference.

### FIELD

At least one embodiment of the present invention lies in the fields of medical technology and computer-aided information processing and relates especially to the checking of a configuration of a complex, multi-component device. In particular misconfigurations are to be detected and other settings or configurations are to be checked for their correctness and for their interaction with one another.

### BACKGROUND

Complex technical systems and devices must generally be set or configured for their intended purpose and subsequent use. For computed tomograph and magnetic resonance tomographs or other medical technology devices it is necessary for example to make a plurality of adjustments or set a plurality of configuration parameters to enable them to be used for the desired purpose and the planned use. In such cases the configurations to be made are partly dependent on one another. This dependency of the configuration settings must be represented in a suitable manner for the user. With today's modern devices this is usually done with computer assistance, so that the configuration settings can be made, analyzed and checked at a user interface.

With previous systems for configuration of a complex device or during checking of such a configuration the user has been guided through a configuration process by means of correspondingly generated displays at a user interface, in order to make the plurality of configuration settings. Previously only a sequential choice of configuration options was possible. In other words the user thus always had to perform the complete sequence of a configuration process from the beginning to the end. It was not possible to determine only part configurations. Furthermore it was also not possible only to check part configurations for correctness.

The previous method is therefore proving very time-consuming and offers little flexibility.

### SUMMARY

At least one embodiment of the present invention is therefore to overcome the disadvantages from the prior art. Furthermore a configuration system with a number of entry points is to be created. Likewise automatic checking of configurations is to be speeded up. Furthermore it is to be possible to carry out the checking of the device configurations of multi-component complex devices in a more targeted manner. In particular unnecessary checks are to be avoided.

A method is disclosed for checking the configurations of a multi-component device by a control computer, by a checking system and by a computer program product.

Features, advantages or alternate forms of embodiment mentioned here are likewise to be transferred to the other claimed subject matter and vice versa. In other words the physical claims (which are directed for example to a system, a facility or a product) can also be further developed with the features which are described or claimed in conjunction with the method. The corresponding functional features of the method are embodied in such cases by corresponding physical modules, especially by a hardware or microprocessor modules.

In accordance with one aspect, an embodiment of the invention relates to a computer-aided method for configuration of a multi-component device and for checking a configuration of the device (which comprises a plurality of device components), wherein the individual device components are able to be configured independently of one another but their dependencies are to be taken into account for the configurations of the device. The method comprises:
Reading in of a graphical representation of a sequential flow diagram or of another representation of a sequential configuration process for all device components of the device to be configured;
Transforming the graphical representation read in into a directed acyclic graph;
Compiling the directed acyclic graph into a set of rules and creating index data for the rules;
Creating a rule-based configuration process logic for interactive configuration of the device with a sequential sequence for configuration of the individual device components and representation of the configuration process logic at a graphical user interface;
When a configuration of at least one device component is changed:
   Detecting at least one following device component which is affected by the change on the basis of the created index data;
   Checking the configuration of the device by dedicated checking of the configuration of the at least one following device component concerned.

A control computer, in at least one embodiment, is connected via a network to further computer-based entities for exchange of data and has a control unit, at least one interface to a central server and a graphical user interface. The graphical user interface serves to display the created configuration process.

A further aspect of an embodiment relates to a checking system and a computer program product.

A storage medium is disclosed in an embodiment, which is intended for storage of the computer-implemented method described here and is able to be read by a computer.

### BRIEF DESCRIPTION OF THE DRAWINGS

In the following detailed description of the figures, example embodiments to be understood as non-restrictive are discussed with their features and further advantages on the basis of the drawing, in which:

FIG. 1 shows an overview-type diagram of a computed tomograph which is to be configured as a device in accordance with an example embodiment of the invention,

FIG. 2 shows a schematic diagram of a control computer which is used the configuration and for checking the configuration,

FIG. 3 shows a schematic diagram of a control computer for checking the device configuration in accordance with an example embodiment of the invention and

FIG. 4 shows a flow diagram of the method for checking a device configuration in accordance with a preferred form of embodiment of the present invention.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Methods discussed below, some of which are illustrated by the flow charts, may be implemented by hardware, software, firmware, middleware, microcode, hardware description languages, or any combination thereof. When implemented in software, firmware, middleware or microcode, the program code or code segments to perform the necessary tasks will be stored in a machine or computer readable medium such as a storage medium or non-transitory computer readable medium. A processor(s) will perform the necessary tasks.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the following description, illustrative embodiments may be described with reference to acts and symbolic representations of operations (e.g., in the form of flowcharts) that may be implemented as program modules or functional processes include routines, programs, objects, components, data structures, etc., that perform particular tasks or implement particular abstract data types and may be implemented using existing hardware at existing network elements. Such existing hardware may include one or more Central Processing Units (CPUs), digital signal processors (DSPs), application-specific-integrated-circuits, field programmable gate arrays (FPGAs) computers or the like.

Note also that the software implemented aspects of the example embodiments may be typically encoded on some form of program storage medium or implemented over some type of transmission medium. The program storage medium (e.g., non-transitory storage medium) may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The example embodiments not limited by these aspects of any given implementation.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

In accordance with one aspect, an embodiment of the invention relates to a computer-aided method for configuration of a multi-component device and for checking a configuration of the device (which comprises a plurality of device components), wherein the individual device components are able to be configured independently of one another but their dependencies are to be taken into account for the configurations of the device. The method comprises:
Reading in of a graphical representation of a sequential flow diagram or of another representation of a sequential configuration process for all device components of the device to be configured;
Transforming the graphical representation read in into a directed acyclic graph;
Compiling the directed acyclic graph into a set of rules and creating index data for the rules;
Creating a rule-based configuration process logic for interactive configuration of the device with a sequential sequence for configuration of the individual device components and representation of the configuration process logic at a graphical user interface;
When a configuration of at least one device component is changed:
   Detecting at least one following device component which is affected by the change on the basis of the created index data;
   Checking the configuration of the device by dedicated checking of the configuration of the at least one following device component concerned.

In other words, an embodiment of the present invention makes it possible to graphically model configuration dependencies or the dependencies of different configuration settings and then convert them automatically into a model, and on the basis thereof, to make it possible to automatically check the configuration. A user can then define or change the corresponding configurations at a control computer (e.g. at a workstation, a PC or a mobile device such as a laptop or a PDA) and perform a corresponding checking run. In this run the dependencies are to be checked for consistency with one another.

An important aspect of an embodiment of the present invention is thus to be seen as converting the previous representation of the configuration sequence—usually in the form of a sequential flow diagram—into a linear constrained based model. A significant advantage is to be seen in the fact that all interdependencies between the respective configurations and configuration settings can be detected and changed automatically.

A further important aspect of an embodiment of the invention includes controlling the configuration process. One of the ways in which this can be done is by making entries at a graphical user interface.

In accordance with a preferred form of embodiment of the invention there is provision for the previous representation of the configuration dependencies in the form of a graphical representation to be converted automatically by a compiler into a constraint-based model. The results of the compiler are transferred into what is known as a constraint solver, which can be used on different platforms. The constraint solver can be accessed for example via Web services and operated via a Web browser.

Advantageously it is possible for the entire device configuration to be able to be checked at any given point in time. Individual configurations or configuration settings can be selected in any given order at any given point in time or released again (deselected).

Preferably there is provision, each time that a change is made in respect of the device configuration, for the change to be checked automatically for permissibility. Unlike in the prior art, the entire device configuration sequence does not always have to be executed completely in the subsequent checking of the device configuration, but instead it is possible to only check the relevant configuration settings for permissibility here. Accordingly the checking measure can be carried out in a significantly more targeted manner, in that only the relevant device components will be checked for permitted configurations.

At each point in time and after each change it is insured that a consistent overall device configuration is always guaranteed. This is important because the configuration of a first device component as a rule has effects on the configuration of at least one further device component.

If for example, in a medical system such as a computed tomograph, it is selected that the patient table is not to be able to be moved, then the configuration option is no longer offered to the user in a subsequent step in which the manner in which the table can be moved is defined, since it is to be stationary in any event. On the other hand only the stationary table can be equipped with a cabled foot switch. The movable table needs a wireless foot switch. Depending on the table size only specific mattresses can be selected.

A further advantage is to be seen in the fact that the configuration of the device and the checking of the configurations made can be carried out in a target group-oriented manner. Thus groups of users can be defined who all operate, configure and/or check the device—possibly in different roles (doctor, MTA, administrator etc.). In such cases configuration wizards or dynamically-generated user interfaces can be generated, which all refer back to the same underlying model.

In accordance with a preferred form of embodiment of the invention, an embodiment of the present invention relates to the primary application of the method in the field of medical technology to the configuration and its checking of imaging devices, such as magnetic resonance tomographs of computed tomographs for example. The embodiment of invention is not restricted to the field of medical technology however, but can also be directed to the configuration of other technical systems having a number of electronic or computer-based components. Since the multi-component complex systems usually demand a plurality of configurations, a purely rule-based is not apposite in this case, since it would entail too much computing power and expense.

It is important that the interdependencies between the individual device component configurations are detected and can be represented in a clear manner, so that they are able to be verified in a simple manner for a user. In such cases a visual representation of the configuration rules can be applied. It is important that the user working at a control computer is guided via the user interface through the respective configuration process or the checking process of the configuration. In this case the user should only be offered a choice of the permissible configuration options, so that he is not burdened by unnecessary configuration options.

At a user interface a sequential execution sequence is created in respect of the configuration; in this case the respective configuration settings are detected and automatically taken into account for the further configuration. Thus all configuration options selected earlier are taken into account for the later configuration run. Then only those permitted further configurations which are consistent with the configuration settings made earlier are ever offered for selection.

A configuration process for a complex (e.g. medical technology) system is a complicated process. To enable the safety and the error-free functioning of the entire system to be configured (or of the respective device) it is necessary for a configuration to always be checked subsequently for permissibility or correctness. Thus as soon as just a single configuration setting is changed (deleted, added or a different setting chosen) it is necessary to have to carry out a checking process for the entire device. In this process all dependencies between the individual configuration settings should be checked. In the prior art it was previously always necessary to completely check the entire configuration process, even if only a very restricted change (e.g. to just one component) has been carried out. In practice this method has proved to be no longer sustainable since it is too time-consuming and the checking process requires too much effort.

The proposed solution provides for the checking process to be able to be now carried out in a targeted manner, in that only specific device components have to be checked for the permissibility of the configuration. For this purpose a choice is made in accordance with an embodiment of the invention as to which components count as “specific” components. Preferably the components defined as “specific components” are those components which are relevant for the respective change and thus are affected by the change. All components that are not affected by the change are irrelevant and are regularly not checked for the checking of the configuration. This enables the checking of the configuration to be carried out in a significantly more dedicated way and more quickly.

In accordance with an embodiment of the invention only the configuration options or configuration settings are thus checked which are affected or influenced by the change of a configuration. For this purpose it is necessary to define the dependencies between the individual configuration components. In accordance with an embodiment of the invention this is a model-based process. In a preferred form of embodiment the modeling and/or the computation of the dependencies and rules of the configuration settings are carried out in a preparation phase. The index data is likewise generated in the preparation phase. Preferably all steps which are executed in the preparation phase are executed on a powerful central server with sufficient CPU capacity.

The actual configuration and the checking of the configuration after a configuration change can then be carried out in the configuration phase. Then in the configuration phase those configuration settings which are affected by the respective change can be found through a single access very rapidly and easily on the basis of the index data. This search and the subsequent checking can thus preferably be carried out locally and also on a low-power electronic device (e.g. on an iPad or on tablets from different manufacturers or on other low-power electronic devices). This represents a significant advantage of an embodiment of the inventive solution. Thus the checking of a complex device configuration can also be carried out on an external low-power control computer. The modeling or the computation of the index data and the dependencies between the configuration settings is done on a powerful computer in the preparation phase.

As already mentioned previously, a particular advantage of an embodiment of the inventive solution lies in the targeted execution of the checking. Following device components not involved are ignored during the checking or no further checks are made on them. This means that there is no validation of their state (e.g. as selected or not selected). By contrast, checks made in accordance with the previous checking mechanisms from the prior art demand a complete checking of the entire configuration, which is associated with significantly higher computing time.

In accordance with a further aspect of an embodiment of the invention the created, rule-based configuration process logic is created such that it can be represented simply at a graphical user interface in a linear sequence. This is preferably done by the creation of check boxes which are presented in accordance with the linear sequence at the interface. The user can then make entries in the corresponding fields in the respective check boxes which are taken into account for definition of the configuration. The user signals made in this way are used to control the further sequence of the configuration process logic. The user is guided through the configuration process by the sequence of check boxes on the graphical user interface. Depending on which configuration settings he makes or which configuration options he selects and/or deselects, the configuration process will be controlled in a modified way. Other configuration options will be offered in the subsequent configuration process. The check boxes are thus created dynamically as a function of the user signal is created in the preceding check boxes. The configuration process logic is thus dynamic and is created as a function of the user inputs of the preceding configurations.

In accordance with a further aspect a set of rules is created automatically which takes account of all interdependencies between the configurations of the individual device components. This automatically enables all mutual dependencies to be taken into account in respect of the configuration for the checking of the overall configuration, without all configuration settings of all components basically having to be checked.

In accordance with a further aspect of an embodiment of the invention, the reading in, transformation, compilation, creation of the configuration process logic, the detection of user signals and/or the checking of a configuration occurs fully automatically. Only the user inputs in respect of selection and deselection are taken into account.

In the created rule-based configuration process logic all conflicts between the individual device component configurations are preferably automatically recognized and also resolved.

A control computer, in at least one embodiment, is connected via a network to further computer-based entities for exchange of data and has a control unit, at least one interface to a central server and a graphical user interface. The graphical user interface serves to display the created configuration process.

A further aspect of an embodiment relates to a checking system and a computer program product.

The terms used in this application will be explained in greater detail below.

The term “device” relates in accordance with the preferred form of embodiment to medical technology devices, such as computed tomographs, MRT systems or other complex and multi-component medical technology systems. Alternate embodiments of the invention can also lie in medical technology fields and relate to non-imaging medical technology systems. Further embodiments relate to the field of automotive technology for product configuration or to other technical fields.

The device components preferably involve electronic components, such as pumps, linear accelerator modules, coils, mechanical devices for moving the patient table, x-ray sources, detectors, optical filters etc. The components are especially identified by having to be configured before use of the respective device. In such cases the individual device configurations are able to be selected or deselected. Depending on the configuration the respective component and/or the entire device can be adapted to be used for a particular purpose. The individual device configurations have dependencies with one another which are to be taken into account during the overall configuration. The components are an element of the device or can be connected to the device as separate components, such as external control modules, external hard disks or other external components for example. Usually the device components are integrated directly into the device.

The configuration process is divided into two time phases:
1. A preparation phase in which the steps of reading in, transformation, compilation and creation of the configuration process logic are carried out and
2. A configuration phase, in which the specified configurations are made or changed, which automatically results in a check on the overall configuration.

The configuration process is usually interactive and is based on the input of user signals for selection and/or deselection of individual configuration options which are offered or presented at the graphical user interface. Depending on the settings the further configuration process is then controlled dynamically. As soon as the change of the configuration or of individual device components has been detected a check on the overall configuration is automatically carried out in order to prevent an inconsistent configuration state existing. A change of a configuration can be carried out by changing a configuration setting, adding or removing a configuration option or by making other changes. “Transformation” refers to the computer-based calculation of a new data structure, namely in a directed, acyclic graph. Thus the data structure has no cycles (acyclic) and has a uniquely defined direction (directed).

The process of “compilation” likewise refers to a conversion of a first data structure into a second data structure, in said case into a set of rules. The rules comprise mandatory configuration settings and optional configuration settings, preconditions and the handling of configuration conflicts. The rules have an “if-then” structure and can also include logically-linked conditions.

The configuration process logic is based on the compiled rules and controls the configuration process. In other words the configuration process logic comprises a temporal execution sequence through the configuration, which takes into account the dependencies of the different configurations (which have been selected by the user). The configuration process logic is thus created dynamically as a function of the user inputs.

The index data is identified by a dataset which is intended to index the respective rule. In this case either the rule as a whole can be indexed, the premise (the condition part) and/or the result part. The indexing serves in an efficient and rapid manner to identify following device components which are affected by a configuration change.

“Following device components” are device components which are affected by a configuration change and must undergo checking in a following process. The following device components are identified from the set of the device components by access to the index data. A number of following device components which are affected by a change can also exist. The set of following device components is a subset of the overall device components of the device to be configured. Following device components are always only defined after a change of a component configuration. In accordance with an embodiment of the invention the following device components are only checked in respect of their configuration status. Those device components which are not affected by the change also not analyzed during the checking process, which has the effect of speeding up the method overall.

A storage medium is disclosed in an embodiment, which is intended for storage of the computer-implemented method described here and is able to be read by a computer.

It is part of an embodiment of the invention that not all steps of the method necessarily have to be carried out on one and the same computer entity, but they can be carried out on different computer entities. The sequence of the method steps can also be varied if necessary.

In addition, it is possible for individual sections of the method described here to be able to be carried out in a saleable unit and for the remaining components to be able to be carried out in another saleable unit—as a distributed system so to speak.

Before a complex multi-component device, which bears the reference number 10 in the figures, is used said device must be subjected to a configuration process. The preferred embodiment of the invention relates to complex medical systems, such as computed tomographs, magnetic resonance tomographs or other complex medical systems for example. Alternate embodiments of the invention can however relate to other technical systems, for example to the field of aerospace technology, automotive technology or manufacturing technology.

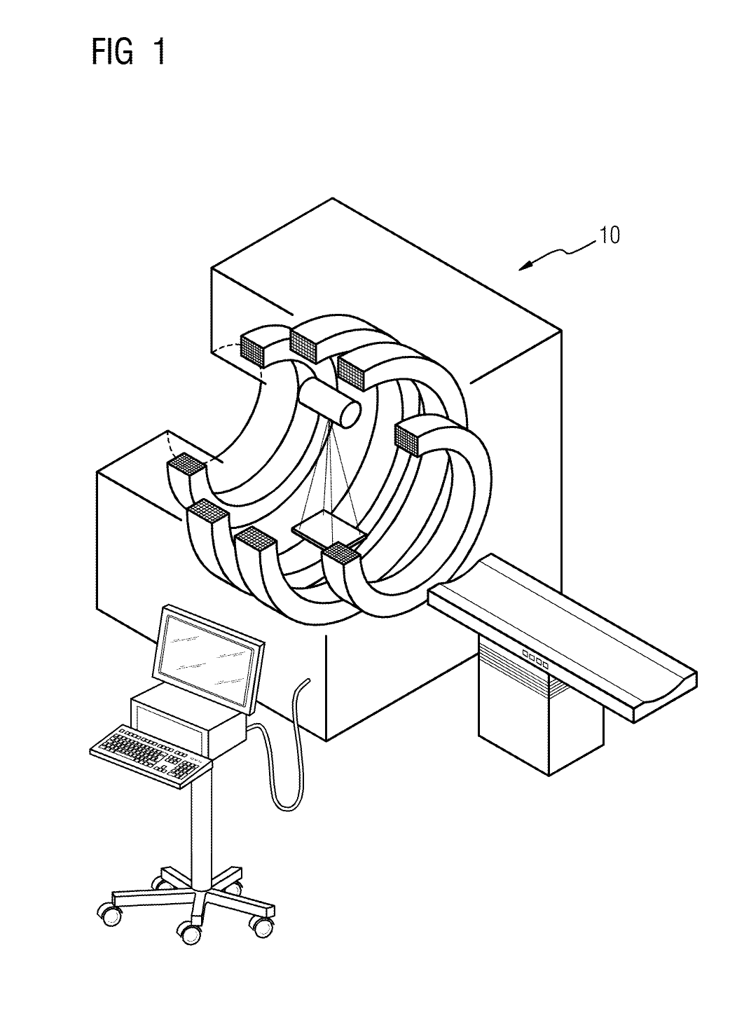

As shown in FIG. 1, a computed tomograph comprises a plurality of technical components K₁, K₂, K₃ . . . K_{N}. The number of the components is basically not restricted. With usual computed tomographs there can be up to 300 components to be taken into account. Components are for example x-ray sources, detectors, coils, the gantry, the patient table, mechanical components etc. The components K can also involve electronic components. In accordance with a further aspect of an embodiment of the invention the components K also include software-based components such as individual software modules, licenses for software modules, versions of software modules etc. These components too must be configured. This means that for example specific program options have to be selected or deselected or set. The program options can relate to a specific functionality of the device 10. In a usual computed tomograph around 700 configuration settings have to be made. The high number of necessary configurations or configuration settings shows that the configuration of the system 10 is not a trivial task, since dependencies between the individual configurations must be taken into account. If for example the user, by making a corresponding user entry, selects a specific design for a detector, in subsequent steps configuration settings in respect of other detectors can be excluded from the configuration process. Only the relevant options are then displayed.

Furthermore it must be ensured that there is always a consistent set of configuration settings available. If for example a user can only select one detector and a first detector can assume the configuration settings a, b, c and a second detector can assume the configuration settings x, y and z, then it would be an inconsistent configuration if the user actually selects the first detector but simultaneously selects the configuration setting y which is only able to be selected for the second detector but not for the first detector. In other words checking of a configuration made must always be carried out. All configuration settings and configuration changes must be checked for permissibility. This is done with an embodiment of the inventive solution automatically and in a very efficient manner.

These dependencies are usually defined in diagrams, known as sequence diagrams. The presentation of the dependencies in the sequence diagrams, especially for complex systems with a plurality of components, is unclear and thus not able to be managed, understood or operated by the user.

With the previous system in the prior art the configuration is undertaken under computer control via inputs which can be made at a user interface of a computer. To this end the user is guided through the configuration process in that a sequential flow diagram representation is presented to him on the screen interface. Depending on previous configuration settings, in accordance with a tree structure, in specific cases the tree structure is branched for the later configuration settings. Specific options can then no longer be selected. A disadvantage of this procedure is to be seen as the user always having to run through the entire configuration tree. Also if he would only like to change and/or check individual configuration settings he needs to run through the configuration tree from beginning to end, which involves a disadvantage as regards time and costs.

In order to avoid this inflexibility, the present invention proposes another method which guides the user through the configuration process in another way. In addition the user can jump into the configuration process at any given points. He no longer needs to carry out the sequential sequence from the beginning to the end.

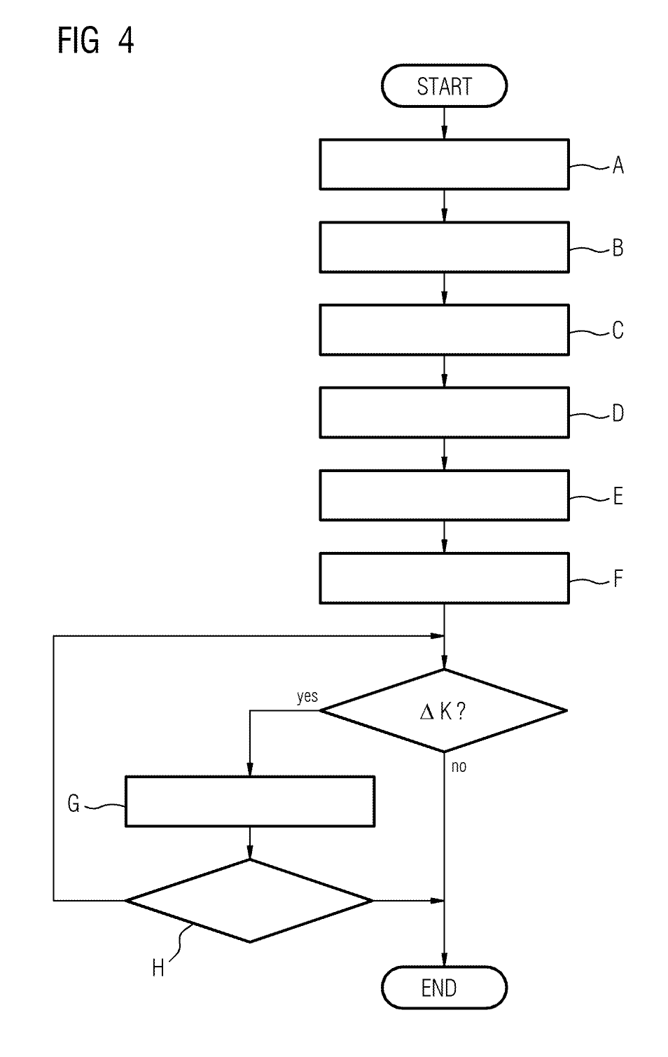

With reference to FIG. 4, a preferred sequence of the configuration and checking process is explained in greater detail.

After the start of the system, in step A a graphical presentation of a sequential flow diagram for configuration of all device components of the device 10 is read in. In a first form of embodiment there is provision to read in the graphical presentation from a memory. In this case the graphical presentation is already available. In a second form of embodiment of the invention the graphical presentation is first created and provided with a graphical tool. In this case the structure created and provided is read in in step A.

Subsequently, in step B the graphical presentation read in is transformed into a directed, acyclic graph. The directed, acyclic graph guides the user through the configuration process and already implements the configuration rules.

In step C the directed, acyclic graph is compiled into a set of rules. Furthermore a file of index data which relates to the set of rules is created in step D. The compilation in step C is carried out via a compiler which converts the data structure into a constraint-based model. The results of the compiler are forwarded to a further module, to the constraint solver and displayed. The rules created relate to the configuration process. In accordance with a first form of embodiment of the invention each rule is indexed. Each of the created rules can be accessed via the index. In a further form of embodiment not only each rule is indexed but also the individual elements of the rules can be indexed. It is thus possible for example to separately index an if part of the rule and/or a then part of the rules. Furthermore the individual elements of the if part and/or the then part can be indexed so that each individual rule term is indexed. This enables access to the individual elements of the configuration rules to be obtained in a very targeted and highly-resolved manner. This is of importance later for the automatic computation of the following device components (in step G).

In step E a rule-based configuration process logic is created. The configuration process logic is used for configuration of the device 10. The configuration process logic is represented at a user interface in order to guide the user through the configuration process. In the preferred form of embodiment this is carried out by displaying check boxes which inform the user about corresponding configuration parameters and then offer him, in accordance with the created configuration process logic, a choice of the appropriate or suitable configuration settings. The user can then select or deselect specific configuration options in a check box. In such cases it is important for the user only to be provided with the choice of options which is permissible based on his previous entries. Inconsistent configuration options are not shown at all, since the created configuration process logic automatically only branches into the permitted cases. The configuration process logic or its representation on the screen interface are used for interactive configuration of the device 10 and lead through the sequential sequence.

As already mentioned, in step F the configuration process logic is represented on the graphical user interface of the control computer KR.

In the subsequent step, which is labeled in the drawing with the reference character ΔK, a check is made as to whether a configuration has been changed. A change can be made by changing an individual configuration setting or changing a number of configuration settings. Furthermore the change can be performed by adding further configuration options by deselecting or removing configuration options. Furthermore it is possible to change certain configuration settings made. The configuration setting, depending on the type of configuration Parameter, can comprise a binary selection (i.e.: yes or no or 0 or 1) or can comprise specifying values (e.g. specifying a number of detectors etc.). In addition yet further input formats can be used for the configuration settings (e.g. also string-type text entries as well as numeric values etc.).

An important advantage of an embodiment of the present invention is now to be seen as the invention making it possible for the user to be able to also make direct changes further down in the configuration process without having to enter the configuration process from the top. As soon as such a change is detected this is automatically recognized by the system and in step G the relevant following device components are computed. Following device components of those components of the device 10 which are affected by the change of configuration. The following device components are detected or computed in a simple manner via the created index data (in step D). If the user for example merely changes the number of x-ray sources then the index data which is assigned to this change is automatically detected in step C and via the index date of the individual rules for the elements of the if parts and/or then parts of the rules are identified which are affected by the change. This enables following device components which are relevant and affected by the change to be identified automatically and without user interaction.

Subsequently, in step H a dedicated check of the following device components that have been identified in step G is undertaken. This represents a significant advantage of an embodiment of the present invention since, when a change is made, the complete configuration with all configuration settings does not—as previously in the prior art—have to be checked (which involves a very slow checking process) but only very few following device components affected by the respective change are checked. The check serves to insure that a consistent configuration process can always be provided. “Consistent” in this context means that the individual configuration settings do not conflict with each other and are permissible. In such cases the interdependencies of the individual configuration settings to one another are also taken into account. Naturally it is also part of the invention here to include formal checking criteria, so that example incorrect or incomplete specifications made by the user are detected and identified. The user is then informed by a further check box being presented to him at the user interface. The criteria and rules which are included for checking the configuration can be defined in a preparation phase. This enables the checking and configuration method to be adapted dynamically to the respective application.

After the checking of the following device components in step H the method can either end or branches are made once again for checking the changes if the user again makes changes to the configuration settings. If he does not make any changes the method can be ended directly.

FIG. 3 shows a schematic diagram of the control of the configuration process or its checking by way of the control computer KR which is controlled by the configuration process logic. The configuration process logic can be a file which is shown for example in the center in FIG. 3 and is stored in a memory MEM. The memory can also involve a database. The configuration process logic includes assignments between the individual device components K₁, K₂, . . . K_{N} and the respective configuration settings KE₁₁, KE₁₂, KE₁₃ for the components K₁ as well as further configuration settings KE₂₁, KE₂₂, KE₂₃, KE_{2N} for the components K₂ and the further configuration settings KE for the respective components K. These assignments are made on the basis of the defined and created rules. All of these rules or assignments are indexed. In such cases it is possible for both the individual components K and also their configuration settings KE to be indexed in order to identify the following device components automatically and rapidly during configuration checking.

A significant advantage of an embodiment of the present invention is to be seen as its ability to enable the control of the configuration process and the checking of a configuration made to be carried out autonomously at a low-power control computer KR.

Furthermore a configuration already made can also be configured and/or checked off-line. The control computer KR does not absolutely have to be connected to a central server.

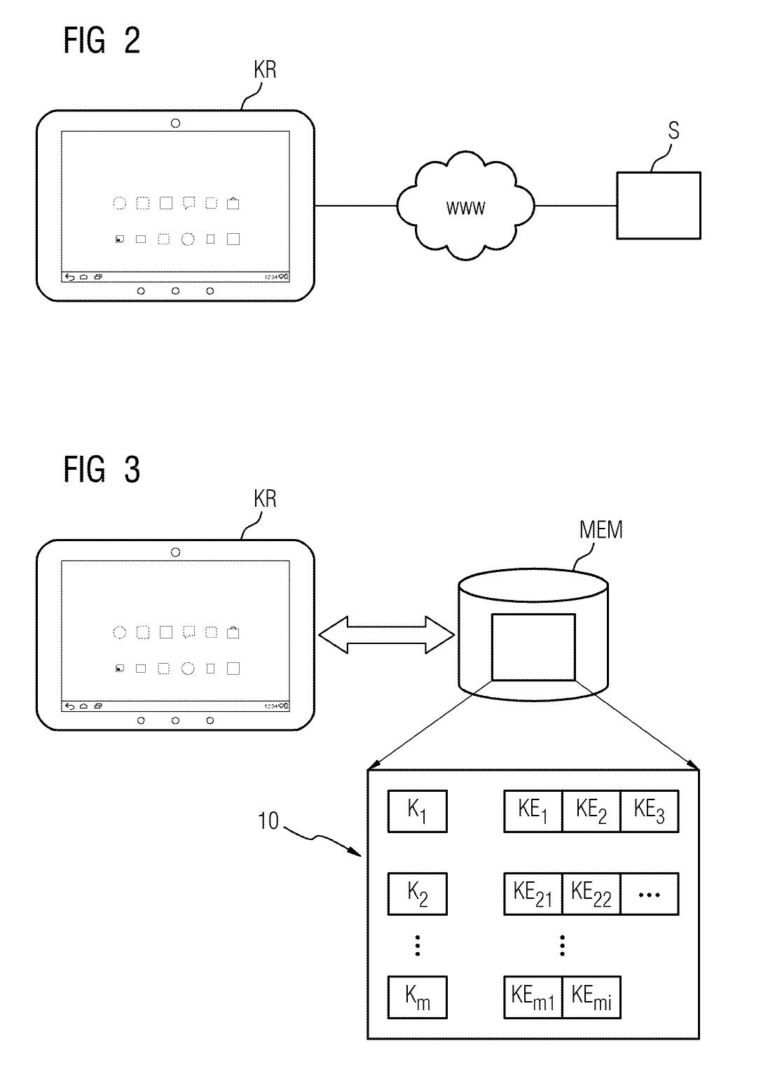

The connection of the control computer KR via a network (e.g. via the Internet) to a central server S shown in FIG. 2 is only an option and is used for updating the configuration rules and/or the index data. For this purpose the control computer KR interacts via a Web server.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for checking a configuration of a medical device with a plurality of device components, individual ones of the plurality of device components being configurable independent of one another, and dependencies of individual ones of the plurality of device components being taken into account for the configuration of the medical device, the method comprising:
reading in a graphical representation of a sequential flow diagram which represents a configuration process for all of the device components;
transforming the read in graphical representation into a directed acyclic graph;
compiling the directed acyclic graph into a set of rules and creating index data for the rules;
creating a rule-based configuration process logic for interactive configuration of the device with a sequential sequence for configuration of the individual device components and representation of the configuration process logic at a graphical user interface; and
if a configuration of at least one of the device components changes
detecting at least one following device component which is affected by the change on the basis of the created index data, and
checking the configuration of the device through dedicated checking of the at least one following device component involved.

2. The method of claim 1, wherein the configuration process logic is represented at the graphical user interface by a linear sequence for presentation of check boxes, wherein the check boxes are intended for detecting user signals and wherein the detected user signals are used for controlling the further sequence of the configuration process logic.

3. The method of claim 1, wherein the created rule-based configuration process logic is created dynamically as a function of each detected user signal for configuration of a device component.

4. The method of claim 1, wherein all the interdependencies between the configurations of the individual device components are taken into account in the set of rules.

5. The method of claim 1, wherein at least one of the reading in, transformation, compilation, creation, detection and checking are carried out fully automatically without user interaction.

6. The method of claim 1, wherein, if a configuration of the device or the device component changes, only the configuration settings of the device components affected by the changes are checked.

7. The method of claim 1, wherein the medical device is a computed tomograph, a magnetic resonance device or another medical technology device.

8. The method of claim 1, wherein conflicts between the individual device component configurations are automatically recognized and resolved in the created rule-based configuration process logic.

9. A control computer for checking a configuration of a medical device with a plurality of device components, individual ones of the plurality of device components being configurable independent of one another, and dependencies of individual ones of the plurality of device components, the medical device being provided separate from the control computer and being able to exchange data with the control computer via a network, the control computer comprising:
a computation unit embodied to carry out at least
reading in a graphical representation of a sequential flow diagram which represents a configuration process for all of the device components,
transforming the read in graphical representation into a directed acyclic graph,
compiling the directed acyclic graph into a set of rules and creating index data for the rules,
creating a rule-based configuration process logic for interactive configuration of the device with a sequential sequence for configuration of the individual device components and representation of the configuration process logic at a graphical user interface, and
if a configuration of at least one of the device components changes
detecting at least one following device component which is affected by the change on the basis of the created index data, and
checking the configuration of the device through dedicated checking of the at least one following device component involved,
an interface to a central server for reading in current index data; and
a graphical user interface to serve to represent the configuration process logic created.

10. A checking system for checking a configuration of a medical device with a plurality of device components, individual ones of the plurality of device components being configurable independent of one another, and dependencies of individual ones of the plurality of device components, comprising:
the control computer of claim 9; and
an interface to a central server for reading in current index data.

11. A computer program product, loadable or already loaded into a memory of a control computer, including commands readable by the control computer for carrying out the method of claim 1 when the commands are executed on the control computer.

12. The method of claim 2, wherein the medical device is a computed tomograph, a magnetic resonance device or another medical technology device.

13. The method of claim 2, wherein conflicts between the individual device component configurations are automatically recognized and resolved in the created rule-based configuration process logic.

14. A non-transitory computer readable medium comprising program segments for executing, when the computer program is executed by a processing unit of an imaging system, the method of claim 1.

15. A non-transitory computer readable medium comprising program segments for executing, when the computer program is executed by a processing unit of an imaging system, the method of claim 2.

16. The method of claim 2, wherein the created rule-based configuration process logic is created dynamically as a function of each detected user signal for configuration of a device component.

17. The method of claim 2, wherein all the interdependencies between the configurations of the individual device components are taken into account in the set of rules.

18. The method of claim 2, wherein the medical device is a computed tomograph, a magnetic resonance device or another medical technology device.

19. A non-transitory computer readable medium comprising program segments for executing, when the computer program is executed by a processing unit of an imaging system, the method of claim 7.
